(19) United States
(12) Patent Application Publication
(10) Pub. No.: US 2026/0306340 A1
(43) Pub. Date: Oct. 8, 2026

(54) ARTICLES AND METHODS FOR ASSISTING MOVEMENT OF EXTREMITIES

(71) Applicant: THOMAS JEFFERSON UNIVERSITY, Philadelphia, PA (US)

(72) Inventors: Mijail Demian Serruya, Merion Station, PA (US); Alessandro Napoli, Philadelphia, PA (US); Mikael Avery, Collingswood, NJ (US)

(21) Appl. No.: 18/998,270

(22) PCT Filed: Jul. 25, 2023
(86) PCT No.: PCT/US23/28552

§ 371 Date: Jan. 24, 2025

Related U.S. Application Data
(60) Provisional application No. 63/392,052, filed on Jul. 25, 2022.
(60) Provisional application No. 63/412,280, filed on Sep. 30, 2022.

(51) Int. Cl. A61H 1/02

(52) U.S. Cl. CPC A61H 1/0288; A61H 2201/0192; A61H 2201/10; A61H 2201/123; A61H 2201/1638; A61H 2201/165; A61H 2201/5007; A61H 2201/5097

(57) ABSTRACT

Provided herein are an upper extremity mobility assistive device and a method of restoring movement of a user's extremity. The upper extremity mobility assistive device includes a main control board; at least one input article coupled to an input of the main control board; and at least one mobility assist element coupled to an output of the main control board, where any input article can be coupled to any mobility assist element. The method includes fitting the upper extremity mobility assistive device to the user's extremity; securing the orthosis device to the user's extremity; receiving at least one input signal at the main control board of the device; and generating at least one output to move the user's extremity. Also provided herein is a method of stimulating movement of a user's extremity using the device disclosed herein.

### CROSS-REFERENCE TO RELATED APPLICATIONS

The present application claims priority under 35 U.S.C. § 119(e) to U.S. Provisional Patent Application Nos. 63/392,052, filed Jul. 25, 2022, and 63/412,280, filed Sep. 30, 2022, which applications are incorporated herein by reference in their entirety.

### BACKGROUND OF THE INVENTION

Introduction. Upper extremity (UE) assistive devices have potential to restore independent, functional movement in people with chronic weakness due to neurological disease and injury. UE assistive devices fabricated in academic settings tend to be impractical in daily life, whereas commercial options are either unaffordable or too challenging to customize.

Accordingly, there is a need in the art for articles and methods that improve on existing extremity assistive devices by providing customizable systems suitable for use in daily life. The present invention addresses this need.

### SUMMARY

In one aspect, the present invention includes an upper extremity mobility assistive device, comprising, a main control board; at least one input article coupled to an input of the main control board; and at least one mobility assist element coupled to an output of the main control board.

In some embodiments, the device further comprises an orthotic brace, the orthotic brace including an arm portion substantially mirroring the shape of a user's forearm; a thumb portion coupled to the arm portion; and a finger portion movably coupled to the arm portion. In some embodiments, the arm portion comprises a top piece and a bottom piece. In some embodiments, the device further comprises a hinge coupling the bottom piece to the top piece in a clamshell fashion. In some embodiments, the thumb portion is coupled to the top piece through the hinge. In some embodiments, the device further comprises a second hinge coupling the thumb portion to the arm portion. In some embodiments, the thumb portion is adjustable relative to the arm portion.

In some embodiments, the device further comprises a hinge movably coupling the finger portion to the top piece. In some embodiments, the hinge coupling the finger portion to the top piece is non-orthogonal relative to the long axis of the user's forearm. In some embodiments, the hinge coupling the finger portion to the top piece is tilted in two planes relative to the long axis of the user's forearm. In some embodiments, the finger portion supports all four of a user's fingers together. In some embodiments, the finger portion leaves a user's fingertips exposed. In some embodiments, the arm portion includes at least one opening leaving a bony prominence on the user uncovered or unimpinged upon. In some embodiments, the bony prominence is selected from the group consisting of the elbow, the scapula, the clavicle, the medial side of the wrist, the lateral side of the wrist, the knuckles, and combinations thereof.

In some embodiments, the main control board comprises a microcontroller; a Bluetooth chip; a motor driver; at least one input connector; a memory chip; and embedded firmware.

In some embodiments, the at least one input article includes an inertial measurement unit (IMU), an electromyography (EMG), a joystick, a switch, a button, a voice recognition module, or any combination thereof.

In some embodiments, the at least one mobility assist element comprises a motor coupled to the arm portion and the finger portion. In some embodiments, the device further comprises a linear actuator coupling the motor to the finger portion. In some embodiments, the at least one mobility assist element comprises a stimulation article. In some embodiments, the stimulation article is selected from the group comprising mechanical vibration, transcutaneous electrical neural stimulation (TENS), functional electrical stimulation (FES), other two-channel electrical stimulation, and combinations thereof. In some embodiments, the stimulation article comprises a functional electrical stimulation (FES) article. In some embodiments, the FES article is arranged and disposed to induce targeted contraction of one or more muscle's in a subject's extremity, the targeted contraction effecting movement of the subject's extremity. In some embodiments, at least one of the stimulation articles is wireless.

In some embodiments, the stimulation article includes at least two stimulation articles; at least one of the stimulation articles is arranged and disposed for positioning on the subject's extremity; and at least one of the stimulation articles is arranged and disposed for positioning over the subject's spine.

In some embodiments, the device further comprises software configured to direct activation of the stimulation article. In some embodiments, the software is configured to set parameters of the embedded firmware. In some embodiments, the software sets the parameters of the embedded firmware to simultaneously activate the at least one stimulation articles arranged and disposed for positioning on the subject's extremity and the at least one stimulation articles arranged and disposed for positioning over the subject's spine.

In another aspect, the present invention includes a method of restoring movement of a user's extremity, the method comprising fitting the orthosis device according to claim 1 to the user's extremity; securing the orthosis device to the user's extremity; receiving at least one input signal at the main control board of the device; and generating at least one output to move the user's extremity. In some embodiments, the at least one output is selected from the group consisting of a powered output through the motor, a stimulation output through the stimulation article, and a combination thereof.

In another aspect, the present invention includes a method of stimulating movement of a user's extremity, the method comprising positioning at least one of the stimulation articles according to an embodiment disclosed herein on the user's extremity; positioning at least one of the stimulation articles according to an embodiment disclosed herein over the user's spine; receiving at least one input signal at the main control board of the device; and generating at least one output to stimulate the user's extremity; wherein the at least one output activates both the at least one stimulation article on the user's extremity and the at least one stimulation article on the user's spine; and wherein the activation of both the at least one stimulation article on the user's extremity and the at least one stimulation article on the user's spine synergistically stimulates the user's extremity.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 shows a schematic illustrating a sequence of study activities in a clinical trial.

FIGS. 2A-B show images illustrating a powered orthosis device, according to an embodiment of the disclosure. (A) A powered orthosis device prior to being donned. (B) A user with the NuroSleeve donned on the paretic left arm and a trigger in the stronger right hand.

FIG. 3 shows an image illustrating a main control unit (MCU) according to an embodiment of the disclosure. The MCU shown includes 2 EMG inputs, 2 IMU inputs and 1 analog joystick.

FIGS. 4A-B show graphs illustrating performance and satisfaction scores on the Canadian Occupational Performance Measure (COPM) for (A) NS1 and (B) NS3.

FIG. 5 shows an image illustrating a powered orthosis device.

FIG. 6 shows a schematic illustrating a trial for evaluating and demonstrating the feasibility of a powered orthosis device.

FIG. 7 shows a schematic illustrating the generation of a 3D printed orthotic brace upon which a motor and microcontroller circuitry can be used to open and close the hand from a 3D scan of a person's forearm.

FIG. 8 shows images of 3D printed components for a powered orthosis that are tailored for a subject's specific finger and hand anatomy.

FIG. 9 shows an image of a power source and input article coupled to a main control unit of a powered orthosis.

FIG. 10 shows a schematic illustrating a subject independently donning a carbon fiber 3D brace.

FIG. 11 shows a subject wearing a soft undersleeve for lining up the position of stimulator electrodes and sensors.

FIGS. 12A-C show images of subject's utilizing a powered orthosis according to an embodiment of the disclosure to complete various tasks. (A) Using a mechanical mini-joystick to trigger hand opening and hand closing. (B) Practicing item stabilization in a paralyzed, motorized hand. (C) Transferring objects with a paralyzed hand using a toe-tap trigger movement via an inertial measurement unit on the contralateral foot.

FIGS. 13A-B show images of an orthosis according to an embodiment of the disclosure. (A) A fully assembled CAD model of a powered orthosis according to an embodiment of the disclosure, prior to a linear actuator being mounted. (B) A fully assembled orthosis, with a linear actuator mounted, donned on a subjects upper extremity.

FIG. 14 shows images illustrating the top (left) and bottom (right) of a printed circuit board according to an embodiment of the disclosure.

FIG. 15 shows images illustrating the top (left) and bottom (right) of a main control board according to an embodiment of the disclosure.

FIG. 16 shows schematics illustrating powered orthosis firmware input/output mapping according to an embodiment of the disclosure.

FIG. 17 shows an image illustrating software UI for a powered orthosis according to an embodiment of the disclosure.

FIG. 18 shows an image illustrating mobility assist device including a stimulation article, according to an embodiment of the disclosure.

FIGS. 19A-D show images illustrating a mobility assist device including bands, according to an embodiment of the disclosure. (A) Left side view of a support brace. (B) Right side view of a support brace. (C) Exploded view of a support brace including a motor and one or more bands. (D) A subject donning a support brace with bands.

### DETAILED DESCRIPTION OF THE INVENTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

Provided herein are articles and methods for providing mobility assistance to a subject's extremity.

### Device

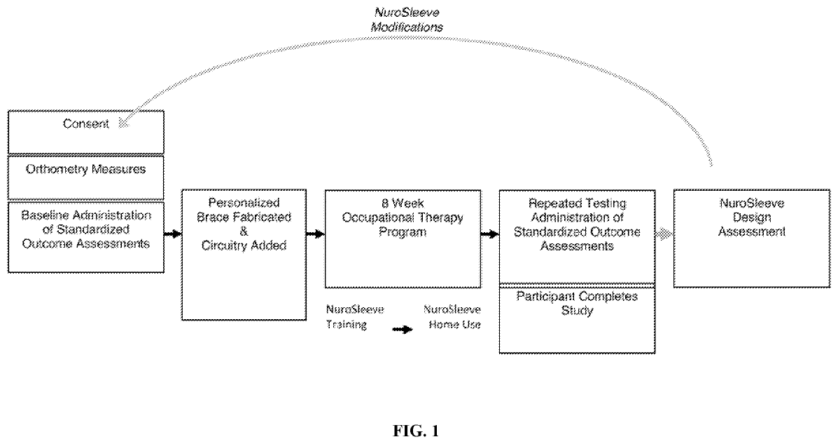

Referring to FIGS. 1-16, in some embodiments, the article is a mobility assistive device 100 including at least one mobility assist element 110, a main control board 120, and at least one input article 130. Each of the at least one mobility assist elements 110 and each of the at least one input articles 130 may be independently coupled to the main control board 120 through any suitable coupling, such as, but not limited to, electrically or wirelessly. As will be appreciated by those skilled in the art, any suitable combination of couplings may be employed, including, but not limited to, wired or electrically coupled input article(s) 130 and mobility assist element(s) 110, wirelessly coupled input article(s) 130 and mobility assist element(s) 110, wirelessly coupled input article(s) 130 and electrically coupled mobility assist element(s) 110, wirelessly coupled mobility assist element(s) 110 and electrically coupled input article(s) 130, or differently coupled input articles 130 (e.g., at least one wirelessly coupled input article and at least one electrically coupled input article) and/or mobility assist elements 110 (e.g., at least one wirelessly coupled mobility assist element and at least one electrically coupled mobility assist element). When coupled thereto, the main control board 120 is configured to receive input from the at least one input article 130 and generate output to the at least one mobility assist element 110.

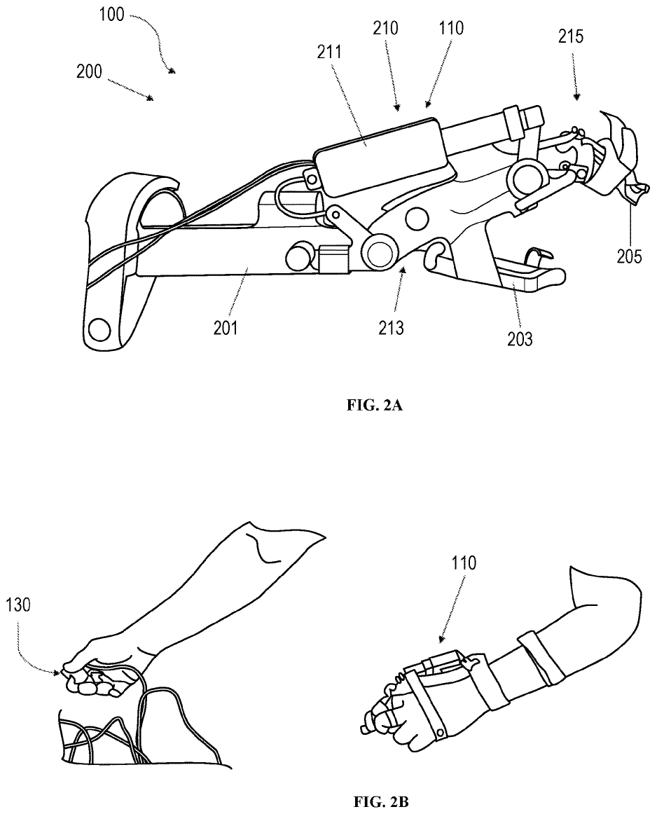

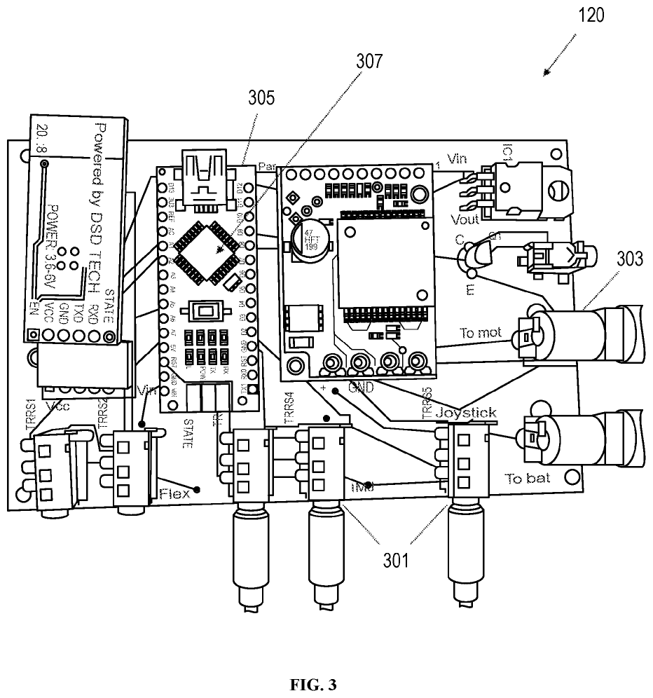

Turning to FIGS. 2A-B, in some embodiments, the at least one mobility assist element 110 includes a movement article 210, such as a motor 211, coupled to a proximal portion 213 and a distal portion 215 of an orthotic brace/prosthesis. The main control board 120 (FIG. 3) is configured to receive input from the at least one input article 130 and generate output to the motor 211. Depending upon the input received from the at least one input article 130, the output generated by the main control board 120 activates the motor 211 to rotate the distal portion 215 in a direction relative to the proximal portion 213. As used herein, the terms “proximal portion” and “distal portion” are defined relative to the motor coupled thereto, and with respect to the subject's core. Therefore, as will be appreciated by those skilled in the art, when more than one motor is present along an extremity, the distal portion relative to a first motor may be the same as the proximal portion relative to the second motor.

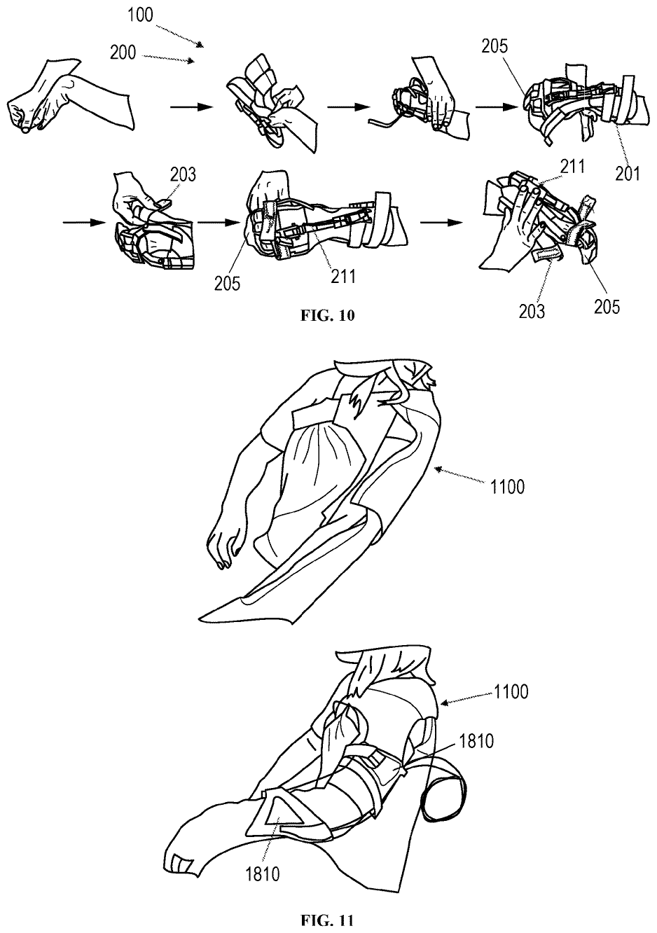

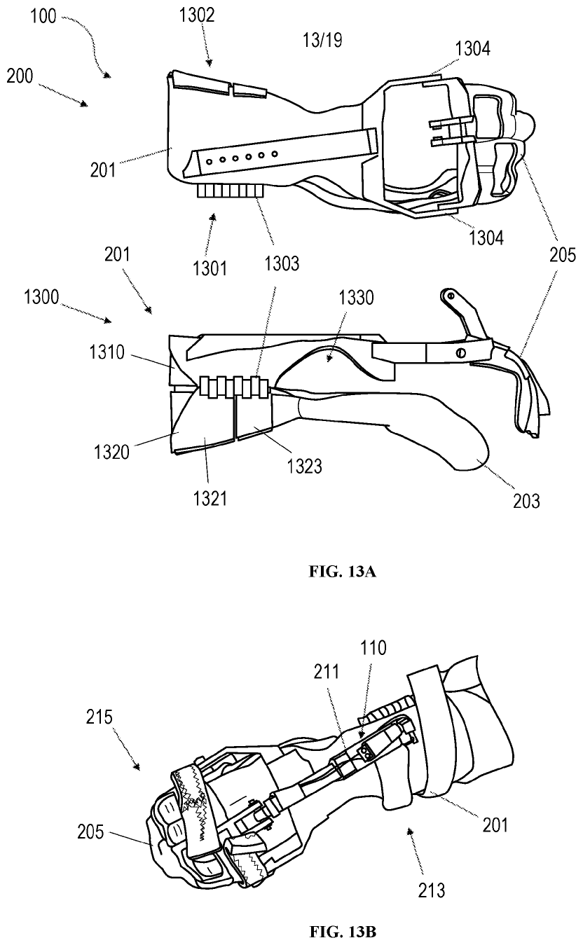

In some embodiments, the device is an upper extremity mobility assistive device 200 including a forearm portion 201, a thumb portion 203 coupled to the forearm portion 201, and a finger portion 205 movably coupled to the forearm portion 201. The forearm portion 201 includes any suitable configuration for donning and doffing the device 200. In some embodiments, for example, as best illustrated in FIGS. 10 and 13A-B, the forearm portion 201 includes a clamshell configuration 1300 (FIGS. 13A-B) with a top section 1310 and a bottom section 1320 connected on a first side 1301 through one or more hinges 1303. In such embodiments, when in the open position, the top section 1310 is first positioned on the subject's forearm, then the bottom section 1320 is closed such that the top section 1310 and bottom section 1320 surround the subject's forearm. After being closed around the subject's forearm, the top and bottom sections are fastened to each other on a second side 1302 to secure the device 200 to the subject's forearm. In some embodiments, the bottom section 1320 includes multiple pieces. For example, in one embodiment, the bottom section 1320 includes a proximal arm piece 1321 and a distal thumb piece 1323. In another embodiment, the separate proximal arm piece 1321 and distal thumb piece 1323 allow the subject to comfortably secure the device 200 to their forearm before accurately placing the distal thumb piece 1323, or vice versa.

In some embodiments, the forearm portion 201 includes at least one opening 1330 corresponding to a bony prominence on the subject. Suitable bony prominences on the subject's forearm include, but are not limited to, the medial side of the wrist, the lateral side of the wrist, the knuckles, and combinations thereof. The opening 1330 includes any gap in material, bump, or other feature that leaves the bony prominence on the user uncovered or unimpinged upon. Without wishing to be bound by theory, it is believed that the opening 1330 corresponding to a bony prominence decreases or eliminates chafing, pressure, discomfort, and/or skin breakdown in those areas, including in subjects with little or no sensation who may be oblivious to such sensations. In addition, the device 200 including these openings 1330 allows opposition of spastic tight limb, fixing of one degree of freedom at the wrist, and providing sufficient rigid support to enable the motor 211 to move the finger portion 205.

The finger portion 205 may be movably coupled to the forearm portion 201 in any suitable manner, such as, but not limited to, through one or more hinges 1304. For example, in some embodiments, the finger portion 205 is coupled to the top section 1310 of the forearm portion 201 through a hinge 1304. In some embodiments, the hinge(s) 1304 is not orthogonal to the long axis of the forearm. In some embodiments, the hinge is tilted in two planes to be the “anatomical average” of the knuckles. This tilting of the hinge 1304 increases the subject's comfort and reduces or eliminates sliding of the subject's fingers relative to the finger portion 205. In some embodiments, the finger portion 205 is configured to move all of the fingers on one of the subject's hands together (e.g., is a single piece configured to secure and move all four fingers on one hand). Alternatively, the finger portion 205 may include multiple pieces configured to move the subject's fingers together or individually.

In addition, the finger portion 205 includes any suitable material or configuration for holding the subject's fingers. For example, in some embodiments, an inner section 701 of the finger portion includes a soft, flexible, and/or elastic material configured to hold the subject's fingers against a comparatively more rigid outer section 703 of the finger portion 205. In some embodiments, the inner section 701 includes one or more pockets for holding the user's fingers. The inner section 701 may be fixed to the outer section 703, such that the subject slides their fingers between the inner and outer section when putting on the device, or the inner section may be detachably coupled to the outer section (e.g., hook and loop fasteners, snaps, buttons, or any other fastening method). In some embodiments, the finger portion 205 is open at a distal end, allowing the subject's fingertips to be exposed. As used herein, the term “exposed” refers to a lack of plastic or other rigid material covering the subject's fingertips, but is not limited exclusively to uncovered fingertips and encompasses flexible materials such as straps or fabric over the fingertips. Without wishing to be bound by theory, it is believed that the exposed fingertips provide improved grasping as compared to synthetic materials that would otherwise cover the fingertips. Additionally, for subjects who retain finger sensation, the exposed fingertips provide improved control.

The motor 211 is coupled to the forearm portion 201 and the finger portion 205 in any suitable configuration for moving the finger 205 portion relative to the forearm portion 201. For example, in one embodiment, the motor 211 includes a motor housing portion 213 and a rod or shaft portion 215 configured to extend from and retract into the motor housing portion 213. In another embodiment, the motor 211 is fixed to the top section of the forearm portion and a distal end of the rod or shaft portion is coupled to a joint or spindle on the finger portion. When the main control 120 board activates the motor 211 to extend the rod or shaft portion 215, the finger portion 205 rotates away from the motor 211 (e.g., towards the subject's palm or to a closed position), whereas when the main control board 120 activates the motor 211 to retract the rod or shaft portion 215, the finger portion 205 rotates towards the motor 211 (e.g., away from the subject's palm or to an open position). Although described herein with respect to a motor 211 and rod or shaft portion 215, as will be appreciated by those skilled in the art, the disclosure is not so limited and includes any other suitable configuration for moving the finger portion relative to the forearm portion. Such other configurations include, but are not limited to, the motor housing portion being fixed to the finger portion and the shaft being coupled to a joint or spindle on the forearm portion, a hydraulic actuator, or any other suitable configuration.

The thumb portion 203 may be movably, adjustably, or permanently fixed relative to the forearm portion 201. When the thumb portion 203 is permanently fixed relative to the forearm portion 201 (e.g., to the top or bottom section), the closing of the finger portion 205 by the motor 211 allows the subject to grasp, squeeze, or hold an object between the finger portion 205 and the thumb portion 203. Similarly, when adjustably fixed relative to the forearm portion 201 (e.g., through a locking hinge), the thumb portion 203 may be positioned for user comfort and/or a desired task, then set to remain in this position during use by the subject. Depending upon the set position, the thumb portion 203 may interact with the finger portion 205 to grasp, squeeze, or hold an object, or the thumb portion 203 may remain clear of the finger portion 205 during opening and closing. Alternatively, when the thumb portion 203 is movably coupled to the forearm portion 201 (e.g., through a hinge), a second motor 211 may be configured to move the thumb portion 203 relative to the forearm portion 201. In such embodiments, the main control board 120 may be configured to move the thumb 203 independently from the finger portion 205, or the thumb portion 203 and finger portion 205 may move in tandem to open and close the subject's hand.

In some embodiments, the upper extremity mobility assistive device is further configured to provide rotation of the subject's hand. For example, in some embodiments, the device includes a separate wrist portion movably coupled to the forearm portion. In such embodiments, the finger portion is movably coupled to the wrist portion, such that the fingers may be moved independent of the rotation of the wrist. The wrist portion may be rotated relative to the forearm portion through any suitable mechanism, such as, but not limited to, a motor. In some embodiments, a first motor is fixed to the wrist portion and coupled to the finger portion, and a second motor is fixed to the forearm portion and coupled to the wrist portion. When the second motor is activated to rotate the wrist portion relative to the forearm portion, the first motor and the finger portion are rotated as well. As such, the first motor may be activated to move the finger portion regardless of the rotation of the wrist portion.

In some embodiments, the upper extremity mobility assistive device includes an upper arm portion. In such embodiments, the upper extremity mobility assistive device may include the upper arm portion without the forearm portion, the upper arm portion coupled to the forearm portion, or the upper arm portion independent from (i.e., not coupled to) the forearm portion. In some embodiments, the upper arm portion extends from any point on the subject's forearm to any point on the subject's upper arm distal to the subject's shoulder. In other embodiments, the upper arm portion extends from any point on the subject's forearm, or any point on the subject's upper arm distal to the subject's shoulder, to a point proximal to the subject's shoulder. Although not shown separately in the figures, as will be appreciated by those skilled in the art, the upper arm portion may be constructed similarly to the forearm portion with dimensions corresponding to the upper arm.

As will be appreciated by those skilled in the art, the upper arm portion may provide movement of the subject's elbow and/or shoulder through any mechanism for moving about a joint disclosed herein. For example, in some embodiments, when the upper arm portion extends to a point proximal to the subject's shoulder, the upper extremity assistive device may include one or more mobility assist elements configured to provide movement about the subject's shoulder. Additionally or alternatively, in some embodiment, the upper arm portion may include one or more mobility assist elements configured to provide movement about the subject's elbow. In such embodiments, the movement about the subject's elbow may be provided without the forearm portion (i.e., no forearm portion present), independent of the forearm portion (i.e., the forearm portion is present but not involved in the movement of the elbow), or in combination with the forearm portion (i.e., the upper arm portion is coupled to the forearm portion to provide movement about the elbow). For example, in some embodiments, when movement about the subject's elbow is provided in combination with the forearm portion, a motor is fixed to the upper arm portion and coupled to a spindle or joint on the forearm portion, the additional motor being configured to move the forearm portion relative to the upper arm portion based upon an output from the main control board (i.e., flex or extend at the elbow).

The upper arm portion includes any suitable configuration for donning and doffing the device, such as, but not limited to, a clamshell configuration similar to that of the forearm portion. Additionally, and once again similar to the forearm portion, the upper arm portion includes at least one opening corresponding to a bony prominence on the subject. Suitable bony prominences on the subject's upper arm include, but are not limited to, the elbow, the scapula, the clavicle, and combinations thereof. The opening includes any gap in material, bump, or other feature that leaves the bony prominence on the user uncovered or unimpinged upon.

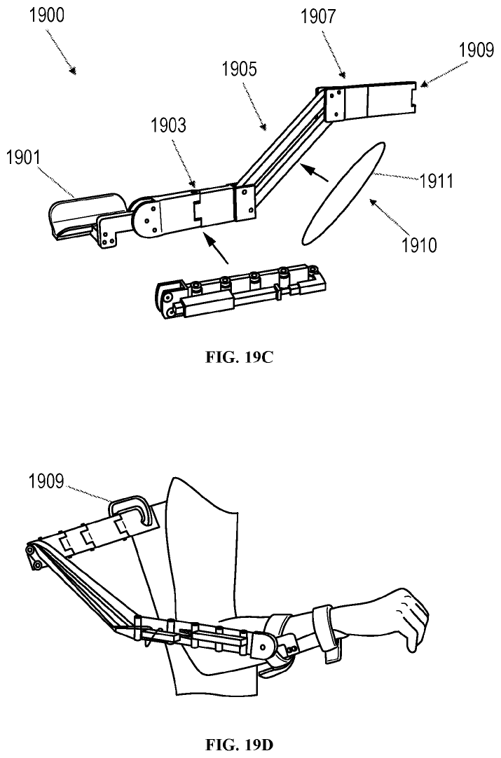

Turning to FIGS. 19A-D, in some embodiments, the mobility assist element 110 includes a passive element 1910, such as, but not limited to, a band 1911. In such embodiments, the bands 1911 may be attached to any suitable brace or support 1900. For example, in some embodiments, the bands 1911 are attached to a brace 1900 configured to support raising, lowering, and/or rotating an upper extremity. As illustrated in FIGS. 19A-B and D, one such brace 1900 includes an extremity support portion 1901 (e.g., an arm support configured for the user to rest their arm in), a joint 1903 (e.g., a forearm/elbow joint that allows for internal rotation), a flexion and extension portion 1905 (e.g., rods that pivot allowing for shoulder), a shoulder piece 1907 that allows for internal rotation at the shoulder level, and a device support portion 1909 (e.g., pieces for attachment to a support, such as a user's chair). In some embodiments, the support brace includes interchangeable blocks that provide a customizable size and/or configuration. Although the passive elements may be used independently, they may also be combined with one or more other mobility assist elements disclosed herein, such as, but not limited to, a motor or stimulation article. In such embodiments, the bands may be attached to one or more of the orthotic braces disclosed herein, or the other mobility assist elements may be combined with the support brace shown in FIGS. 19A-B, as illustrated in FIG. 19C.

Any suitable band or bands having a spring constant sufficient to support the desired extremity movement may be used as passive mobility assist elements 1910. In some embodiments, one or more bands may be engaged and/or disengaged as desired. The engaging/disengaging may be through any suitable mechanism, such as, but not limited to, a button, switch, lever, knob, or other suitable control. This selective engaging/disengaging of one or more bands provides variability and control over the amount of support provided by the device, further helping the subject to move their extremity. Additionally or alternatively, in some embodiments, one or more of the bands may be semi-passive, such as, for example, an electroactive polymer band. In such embodiments, an external trigger may be used to modulate one or more properties of the band (e.g., change their spring constant).

### Stimulation Elements

In some embodiments, the at least one mobility assist element 110 includes a stimulation article, other movement article, and/or biofeedback article (e.g., physical and/or visual feedback) (collectively 1810) coupled to the main control board 120. Suitable stimulation articles 1810 include, but are not limited to, a mechanical vibration article (e.g., motor with an eccentric mass or a vibrating diaphragm), transcutaneous electrical neural stimulation (TENS), functional electrical stimulation (FES), other two-channel electrical stimulation, other stimulation articles, or a combination thereof. Any suitable stimulation article 1810 according to one or more of the embodiments disclosed herein may be configured to induce muscular contraction and movement; to reduce spasticity, relieve pain, and/or help stretch; provide feedback to the user; or a combination thereof. In some embodiments, configuring the stimulation article to induce muscular contraction and movement includes positioning the stimulation article over a specific location on the subject's extremity such that the output from the stimulation article is directed to, and induces contraction in, a particular muscle or muscles that produce a desired movement. For example, an FES article may be positioned such that the electrical output from the FES article induces contraction of muscles in the subject's forearm, which results in flexion of the subject's hand.

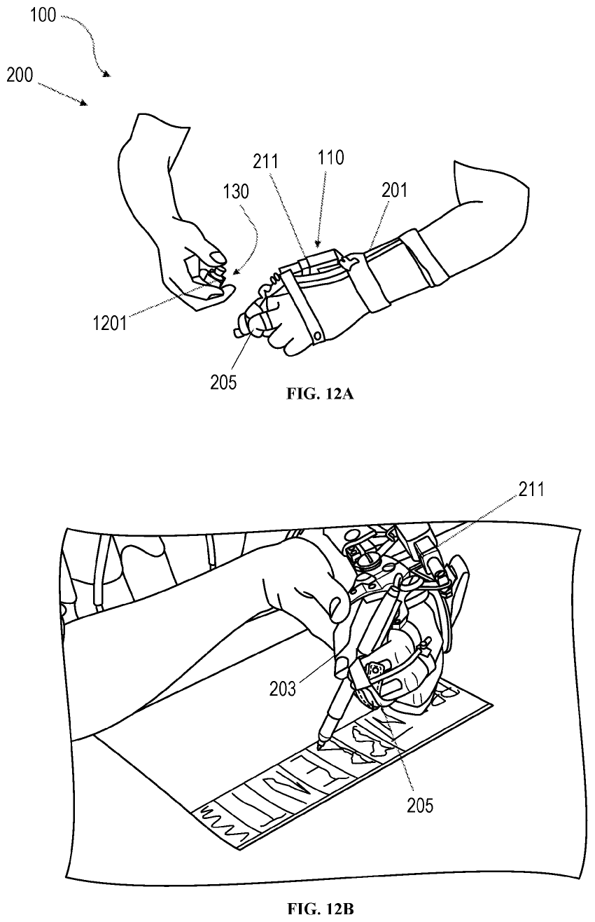

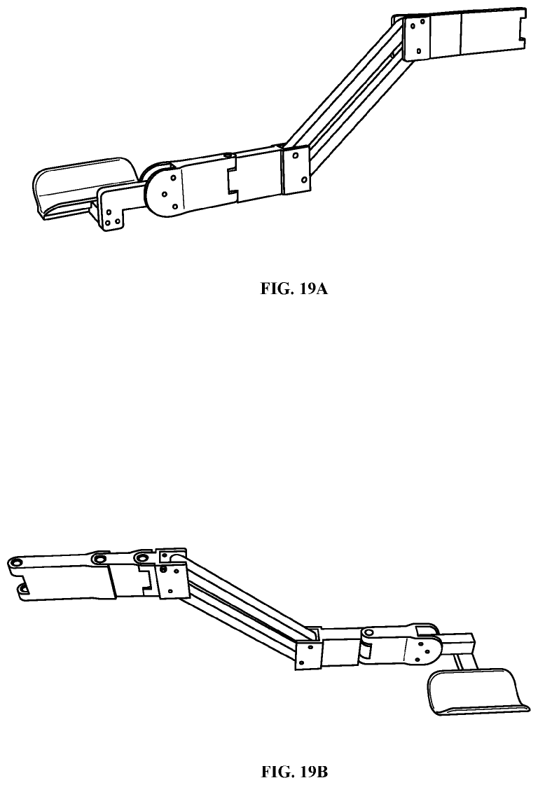

In some embodiments, the stimulation article is applied and/or positioned using any suitable positioning mechanism 1100, such as, but not limited to, a fitted sleeve, wraps, elastic bands, the brace according to one or more of the embodiments disclosed herein, or any other suitable mechanism for guiding the placement of the stimulation article. For example, in some embodiments, as illustrated in FIG. 11, the stimulation article may be placed using a fitted sleeve 1101 that is positioned over the user's extremity. After being placed, the stimulation article 1810 may be held by the positioning mechanism 1100 or secured directly to the user through any suitable element, such as, but not limited to, adhesive pads. Alternatively, in some embodiments, as illustrated in FIG. 18, the stimulation article 1810 is applied and/or positioned directly on the subject, without the positioning mechanism 1100. Any suitable number of stimulation articles 1810 may be positioned on the user, each of which may be individually controlled by the main control board 120 or controlled in combination with one or more other stimulation articles 1810. The stimulation articles 1810 may be connected to the main control board through wires or wirelessly.

In some embodiments, the stimulation article 1810 is used in combination with the brace and/or motor disclosed herein. In such embodiments, the stimulation article may be fixed to the brace such that the article is positioned when the brace is donned. Additionally or alternatively, one or more of the stimulation articles may be positioned independently of the brace. For example, one or more of the stimulation articles may be positioned using the fitted sleeve, or placed directly on the user, and then the brace may be donned over the positioned stimulation articles. When combined with the motor, the stimulation article may simultaneously and/or separately support movement provided by the motor, provide a secondary movement, and/or provide a non-movement function (e.g., reduce spasticity, relieve pain, help stretch, provide feedback, etc.). For example, the stimulation article may induce contraction in a muscle corresponding to a movement being provided simultaneously by the motor (e.g., contraction in the muscles involved in flexion of the hand, elbow, or shoulder as the motor provides flexion). In another example, the stimulation article may separately induce contraction in a muscle opposite a movement provided by the motor (e.g., contraction in the muscles involved in extension of the hand, elbow, or shoulder before or after the motor provides flexion). In a further example, the stimulation article may sequentially and/or simultaneously induce contraction in a muscle distinct from a movement provided by the motor (e.g., contraction of the muscles involved in rotation of the wrist before, during, or after and extension/flexion provided by the motor).

Alternatively, in some embodiments, the stimulation article is used separately and/or independently from the brace and/or motor disclosed herein. As will be appreciated by those skilled in the art, when positioned without a brace or other support structure, the stimulation article may be the primary and/or only form of mobility assistance. For example, the stimulation article may be positioned directly on the forearm, without a brace or motor, to independently provide movement of an extremity through induced contraction of a particular muscle or muscles. In some embodiments, multiple stimulation articles are positioned on the subject to provide different movements (e.g., rotation and flexion) and/or opposing movements (e.g., flexion and extension).

In some embodiments, one or more of the stimulation articles may be positioned on the user in a location separate from the extremity being moved. For example, in some embodiments, the stimulation articles are positioned over a user's spine. In such embodiments, the stimulation articles may be arranged and disposed to stimulate the spine in a location that corresponds to the extremity being moved (e.g., where the nerves from the extremity being moved innervate in the spine). In some embodiments, the stimulation at the spine provides a synergistic effect with the movement of the extremity, whether such movement is through separate stimulation at the extremity or through the brace/motor.

### Device Control

Whether through the motor, the stimulation article, or a combination thereof, the active movement of a subject's extremity by the device according to any of the embodiments disclosed herein is controlled by the main control board 120 (FIGS. 3 and 14-15). The main control board 120 includes at least one input connector 301 (e.g., plug, receptacle, wire, wireless receiver, etc.), at least one output connector 303 (e.g., plug, receptacle, wire, wireless receiver, etc.), a microcontroller 305, a memory chip 307, and embedded firmware. The embedded firmware is stored on the memory chip 307, and is executed by the microcontroller 305 to provide an output signal to the mobility assist element 110 through the at least one output connector 303 based upon an input received from the input article 130 through the at least one input connector 301. Any suitable input article or combination of input articles may be connected to the at least one input connector of the main control board to provide the input signal(s). Suitable input articles 130 include, but are not limited to, an inertial measurement unit (IMU), an electromyography (EMG), a joystick 1201, a switch, one or more push-buttons, a capacitative button, a force-sensitive resistor, a strain gauge, a voice recognition module, an implantable medical device (e.g., decoded signals from a brain implant), an air pressure monitor/gauge, a video module for tracking eye gaze, or combinations thereof. Once an input signal is received from the one or more input articles, the main control board generates one or more output signals according to the firmware stored on the memory chip. These output signals are then communicated to one or more of the mobility assist elements through the at least one output connector.

Any desired output signal or signals may be generated in response to any suitable input signal or signals received by the main control board. For example, in one embodiment, the main control board generates one or more output signals that activate a motor and/or stimulation article of the device to move the finger portion in response to one or more input signals from a joystick. In another embodiment, the main control board generates one or more output signals that activate a motor and/or stimulation article of the device in response to one or more input signals from a voice recognition module and/or an implantable medical device. As will be appreciated by those skilled in the art, the disclosure is not limited to the inputs and outputs in the examples above and may include any other suitable combination of inputs and outputs. Other suitable inputs and outputs include, but are not limited to, inputs from multiple input articles generating a single output (e.g., both the IMU and joystick effect movement of the finger portion), inputs from a single input article generates multiple outputs (e.g., inputs from the joystick effect both activation of a motor and a stimulation article, or activation of multiple motors/stimulation articles), multiple inputs generate multiple outputs (e.g., inputs from the joystick and the implantable medical device effect both activation of a motor and a stimulation article, or activation of multiple motors/stimulation articles). Accordingly, the device may be configured to provide only powered orthosis, only stimulation, or both. In some embodiments, the powered orthosis and stimulation work together synergistically, such as where motor restores one type of action (e.g., extending fingers) and FES restores another type of action (e.g., supination of the forearm, or extension of the elbow).

In some embodiments, the microcontroller is programmed to distinguish between true triggers and false positives. For example, in some embodiments, the microcontroller is configured to detect when a user is walking versus sitting. In some embodiments, the sensors that provide the input and/or one or more other sensors for detecting motion/positioning provide gather and provide data to determine the user's activity. The ability to discern between walking and sitting enables the device to utilize various inputs (e.g., inertial) without triggering unintentional movement of the device while the user is performing certain activities (e.g., walking). Additionally or alternatively, the microcontroller may be programmed to track and/or detect a user's posture using the sensors.

### Software/Firmware

In order to generate a desired output signal or signals, any suitable firmware may be stored on the memory chip. In some embodiments, for example, the firmware includes that disclosed in International Application No. PCT/US2021/041087, which is incorporated herein by this reference. As will be appreciated by those skilled in the art, the disclosure is not so limited and may include any other suitable firmware for providing a desired output. Additionally or alternatively, in some embodiments, the device includes software for configuring the firmware, monitoring use and progress, communicating and/or storing data (e.g., use, progress, issues), and/or facilitating any other communication with the device. In some embodiments, for example, the software includes that disclosed in International Application No. PCT/US2021/041087, which is incorporated herein by this reference.

In some embodiments, the software enables an individual to update/configure the firmware to modify the output effected by any one or more inputs received (e.g., to link any input to any output). The individual (e.g., a therapist) may then lock the configuration of the firmware so a user can simply use the device as configured. For example, an input initially configured to move the finger portion may be modified to change the output (e.g., rotate the wrist portion), add one or more additional outputs (e.g., add stimulation), switch to another input (e.g., IMU), add one or more additional inputs (e.g., add a second input that also generates an output to move the finger portion), or a combination thereof. In some embodiments, the data communicated or stored through the software is used to further configure the device.

In some embodiments, the software enables an individual to store multiple settings/configurations, facilitating quick/easy switching between the settings/configurations. In some embodiments, the switching is done by the individual through the software. Additionally or alternatively, in some embodiments, the switching is effected through a mechanism accessible to the user that switches between stored configurations in order to modify the input and/or output settings. In some embodiments, the stored configurations correspond to different uses for different activities. For example, in some embodiments, the device includes a button that activates and deactivates IMU as a secondary input for moving the finger portion, switches from joystick to IMU for moving the finger portion, or adds or removes any other input or output.

The software may be stored and/or executed on the device or on a separate interface device. For example, a monitor may be connected to the device in order to visualize/access the software stored on the device, or a computer/tablet/other interface device including the software may be connected to the device to enable configuration/setting of the firmware and/or other communication with the device. As will be appreciated by those skilled in the art, the monitor and/or separate interface device may be connected to the device through any suitable mechanism, such as, but not limited to, wired or wirelessly. Accordingly, in some embodiments, the device includes a wireless connection mechanism such as, but not limited to, wifi, Bluetooth, or any other suitable wireless connection mechanism. The wireless connection mechanism provides two-way communication with the device, providing a healthcare professional or other authorized third party with the ability to modify the firmware remotely, monitor use and progress, and otherwise communicate data to and/or receive data from the device.

### Telemedicine/Device Monitoring

In some embodiments, the data storage and/or communication capability of the device permits monitoring of the device. For example, in some embodiments, the device communicates data relating to device use and/or user feedback to a medical professional, such as a therapist. This data may include frequency of use, specific movements performed, and/or user comfort to track progress (rehab) and/or inform modifications to the device configuration. In some embodiments, the modifications and/or tracking may be performed remotely, facilitating telemedicine application of the device. Additionally or alternatively, the information may be communicated to the user, such as through an integrated personal device (e.g., smart phone, tablet, etc.), enabling direct monitoring of progress. Specific exercises may also be provided to the user through the integrated personal device, further enabling tailoring and monitoring of care.

### Methods of Making and Using Device

Also provided herein are methods of making and using the device according to one or more of the embodiments disclosed herein. In some embodiments, the method includes fitting the device to the subject's extremity. For example, the method may include adjusting an existing orthosis device to fit the subject's forearm. Alternatively, in some embodiments, the method includes obtaining a three-dimensional (3D) scan of the subject's extremity and producing a customized orthosis based upon the 3D scan. The customized orthosis may be produced by any suitable method, such as, but not limited to, 3D printing, molding, or any other suitable method. It was surprisingly found that for some subjects, an orthosis that fits too well is uncomfortable to wear. Accordingly, in some embodiments, the method includes producing an initial orthosis based upon the 3D scan, assessing the fit of the initial orthosis, adjusting the design according to the fit (e.g., increasing the internal size to accommodate additional padding or provide a desired amount of snugness), and producing a second orthosis based upon the adjusted design. After fitting the orthosis to the subject's extremity, the method optionally includes adjusting one or more of the inputs or outputs to provide the subject with a customized operation. For example, the inputs may be modified to push button control of the finger portion instead of joystick control, to add IMU control, to add control from an implantable device, or to otherwise modify the input controls. Similarly, the outputs may be modified to change the speed or range of motion of the device (e.g., finger portion), add an output (e.g., stimulation or biofeedback), change an output, or a combination thereof.

In some embodiments, the method includes training the subject on the device and/or modifying the operation based upon the subject's individual use of the device. For example, in some embodiments, the firmware may be updates to better distinguish between true triggers and false positives, or to change the sensitivity of the inputs or parameters of the outputs (e.g., speed, range of motion). Additionally or alternatively, in some embodiments, the training includes activating one or more biofeedback mechanisms on the device. In one embodiment, for example, the biofeedback includes physical feedback, such as vibration or stimulation. This may be activated when a motion is activated to help the subject learn to exercise the cognitive circuits and residual control abilities in a way that would otherwise be impossible. In another example, the biofeedback includes a visual output, such as, but not limited to, a “toy” output for pediatric subjects. The toy may be any type of visual output, including, but not limited to, a soft-dart launcher on the forearm orthosis of the pediatric subject. In such embodiments, the pediatric subject can launch the soft dart from the device, which not only provides entertainment to increase use, but also helps the subject learn to exercise the cognitive circuits and residual control abilities in a way that would otherwise be impossible.

The devices and methods disclosed herein are capable of providing any suitable subject with assistive mobility. Suitable subjects include, but are not limited to, those with hemiparesis due to neurological disease or injury. Without wishing to be bound by theory, it is believed that the devices and methods disclosed herein restore independent, functional movement to accomplish everyday tasks beyond the rehabilitation clinic. Additionally, the devices and methods disclosed herein provide increased usability, comfort, and functionality through the personalized shape, type of input source, location of the input source (e.g., arm, shoulder, leg), and/or type of output.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

### Example 1

Stroke, spinal cord injury, muscular dystrophy and other neurological conditions frequently cause debilitating upper extremity (UE) motor impairments that last beyond rehabilitation discharge. UE assistive devices have potential to restore independent, functional movement in people with chronic weakness due to neurological disease and injury. However, UE assistive devices fabricated in academic settings tend to be impractical in daily life, whereas commercial options are either unaffordable or too challenging to customize. For example, although several passive and powered UE braces have been developed, the only commercially available wearable, powered arm orthosis is the MyoPro system (Myomo, Inc, Cambridge, Massachusetts). The MyoPro features a myoelectric approach in which continuously recording surface electromyographic signals trigger motors to flex and extend the fingers or elbow into the desired position. This myoelectric strategy is adopted from prosthetics, where myoelectric control has been integrated into prosthetic limb designs for over 70 years (P. B. Nicholls, D. A. Stevenson, E. D. Sherman, A. L. Lippay, and G. Gingras, “A Canadian electric-arm prosthesis for children.,” Can. Med. Assoc. J, 1967; L. McLean and R. N. Scott, “The Early History of Myoelectric Control of Prosthetic Limbs (1945-1970).,” in Powered Upper Limb Prostheses, A. Muzumdar, Ed. BerlinHeidelberg: Springer-Verlag, 2004, pp. 1-2.). Multi-week regimens incorporating the MyoPro portable myoelectric elbow-wrist-hand orthosis as a therapeutic adjunct to rehabilitative therapy have been shown to increase UE function, use, and recovery in individuals with stroke, with moderate UE deficits (S. J. Page, V. Hill, and S. White, “Portable upper extremity robotics is as efficacious as upper extremity rehabilitative therapy: a randomized controlled pilot trial.,” Clin. Rehabil., vol. 27, no. 6, pp. 494-503, 2013; G. J. Kim, L. Rivera, and J. Stein, “Incorporating a wearable upper extremity robotics device into daily activities at home: A case series,” Arch. Phys. Med Rehabil., vol. 94, no. 10, pp. e33-e34, 2013; A. Ramanujam et al., “Poster 64 Incorporating a Wearable Upper Extremity Robotics Device into Daily Activities at Home: A Case Series,” Arch. Phys. Med. Rehabil., vol. 94, no. 10, pp. e33-e34, October 2013; H. T. Peters, S. J. Page, and A. Persch, “Giving Them a Hand: Wearing a Myoelectric Elbow-Wrist-Hand Orthosis Reduces Upper Extremity Impairment in Chronic Stroke,” Arch. Phys. Med. Rehabil., 2017.).

The NuroSleeve system developed in this Example is a wearable lightweight, low-profile, patient-tailored forearm brace that can use as inputs electromyography (EMG), inertial measurement units (IMU), and an external control source (i.e., joystick) to control the movement of a motor, which in turn facilitates hand open-close movement. Hence the NuroSleeve is customizable in both form and function: the structure of the brace is personalized to the patient's forearm shape, the occupational therapist can select both the type and location of the input source. Through an eight-week rehabilitation program designed to include occupational therapy and experimental home use of the device, both the feasibility of the NuroSleeve and the iterative design process incorporating feedback from occupational therapists, engineers, physicians, industrial designers and the two individuals using the device are outlined herein.

As discussed in detail below, two adults with UE mobility impairment due to stroke were able to learn to use a lightweight, customized, powered hand orthosis/prosthesis. Providing the hand therapist and the user the ability to trigger hand motion with mechanical and inertial switches facilitated enhanced usability to perform activities of daily living.

Methods Two individuals with hemiparesis from chronic stroke participated in an eight-week intervention with the NuroSleeve.

### Participant Characteristics

NS1 is a 41-year-old, left-handed male veteran of the United States military with dense left hemiparesis and dysarthria following a large right sided spontaneous basal ganglia hemorrhage seven years prior (I61.01, G81.1). In the year prior to enrollment in the NuroSleeve trial, the participant was prescribed the MyoPro powered upper extremity orthosis; he found this difficult to don, doff and use and stopped using it within a few months of receiving it.

NS3 is a 53-year-old right-handed male with left hemiparesis that arose due to an ischemic stroke in the right middle cerebral artery territory due to dissection and occlusion of the right internal carotid artery, four years prior to enrollment in this trial (I720.0, I63.4, G81.1). His post-stroke recovery was complicated by agitation that led to falls that in turn led to skull fractures: this is mentioned because it is likely that some degree of traumatic brain injury occurred and was superimposed upon the stroke and this may have contributed to alterations in cognition, processing speed and coordination of motor control. Due to his inability to perform numerous activities of daily living and high motivation to perform additional therapy, he was offered a powered orthotic brace, the MyoPro, to assist with left arm functionality. However, due to concerns that the weight of the MyoPro might exacerbate post-stroke shoulder subluxation, therapy with MyoPro was not pursued.

### Intervention

The 8-week intervention consisted of the NuroSleeve wrist-hand orthosis, out-patient occupational therapy, home use, and is described in detail here.

The sequence of study activities is shown in FIG. 1. Following consent, standardized outcome assessments were administered. Next, the participants were provided a custom fit NuroSleeve device. Following 8 weeks of outpatient occupational therapy, the standardized outcome assessments were repeated.

The outpatient occupational therapy program consisted of three, 45-minute sessions each week over an eight-week period. Prior to beginning therapy, the Canadian Occupational Performance Measure (COPM) (M. Law, S. Baptiste, M. Mccoll, A. Opzoomer, H. Polatajko, and N. Pollock, “The Canadian Occupational Performance Measure: An Outcome Measure for Occupational Therapy,” Can. J. Occup. Ther., 1990) was administered to identify 3-5 activities in multiple domains (including work, self-care and leisure) that the participants wanted, needed or were expected to do, but were either not doing or were not satisfied with how they were doing them. While those activities served as the goals for the NuroSleeve and occupational therapy, principles of motor learning and motor control (M. Maier, B. R. Ballester, and P. F. M. J. Verschure, “Principles of Neurorehabilitation After Stroke Based on Motor Learning and Brain Plasticity Mechanisms,” Front. Syst. Neurosci., vol. 13, December 2019.) and task-specific activity-based therapy (D. Backus, “Activity-Based Interventions for the Upper Extremity in Spinal Cord Injury,” Top. Spinal Cord Inj. Rehabil., vol. 13, no. 4, 2008; N. Grampurohit et al., “Highlighting gaps in spinal cord injury research in activity-based interventions for the upper extremity: A scoping review,” NeuroRehabilitation, vol. 49, no. 1, pp. 23-38, 2021.) with and without the NuroSleeve were incorporated into each session. Training in the use of the NuroSleeve for completing the COPM goals was also provided, initially during therapy under the direction of the therapist and, once comfortable with the device and deemed ready to do so, unsupervised at home. The same licensed occupational therapist, who has 8 years of experience in neurorehabilitation of the upper limb, implemented the 8-week program with both participants.

The SCI Physical Therapy and Occupational Therapy Basic Data Set (K. AU—Anderson et al., “International Spinal Cord Injury Physical Therapy—Occupational Therapy Basic Data Set (Version 1.2),” Spinal Cord Ser. Cases, vol. 6,2020.) was used to record intervention details. The participants attended all planned sessions (there were no missed therapy appointments). After completion of their participation in the study, participants were allowed to keep their personalized device.

### NuroSleeve

The NuroSleeve is a powered orthotic brace that weighs less than half a pound and can use as input any one or more of the following: 1) surface EMG signals from the biceps, triceps, brachioradialis and wrist extensors 2) IMU signals from the shoulder, leg, or other location convenient for the user, or 3) manual control using a variety of control sources. Each participant worked with the interventionist occupational therapist to identify a suitable control mode.

The NuroSleeve is comprised of the “Flex Hinge Wrist Hand Orthosis” (Jaeco Orthopedic, Hot Springs, AR) with a 1 inch-stroke linear actuator motor model PA-07 (Progressive Automations, Arlington, WA) mounted to it and control circuitry, specifically designed and developed by the authors. The NuroSleeve Main Control Unit (MCU) consists of 1) an Arduino Nano platform (Arduino, Italy) for running the real-time control firmware, handling the sensors, signal processing, closed-loop control, and managing motor control outputs; 2) a Bluetooth chip DSD HC-05 (DSD Tech, China) for communication with local PC; 3) a Motor Driver Carrier VNH5019 (Pololu Robotics and Electronics, Las Vegas, NV); 4) EMG sensors MyoWare (Sparkfun, Niwot, CO); and 5) IMU sensors BNO055 (Adafruit, New York City, NY). The NuroSleeve also features in-house developed Windows software for exchanging data with device, as well as managing and setting all parameters of the embedded device firmware. The Hinge Wrist Hand Orthosis is made of stainless steel that is hammered by a skilled technician at Jaeco Orthopedic, to match the dimensions of the individual person's forearm and hand based on measurements specified by the freely downloadable Orthometry Form (“JAECO Orthometry Form.” [Online]. Available: jaecoorthopedic.com). The powered Hinge Wrist Hand Orthosis is shown in FIGS. 2A-B, while FIG. 3 shows the NuroSleeve Main Control Unit assembly.

### Standardized Assessments

A battery of standardized outcome assessments was administered prior to and following the intervention period. At follow-up, each measure was administered with and without the NuroSleeve. One of two trained occupational therapists, neither of whom provided the occupational therapy program, administered the standardized outcome assessments; both of these therapists were certified and experienced in the administration of these outcome measures.

Participants' COPM goals were rated on an ordinal scale between “1” (cannot do/not satisfied) and “10” (performs well/very satisfied COPM). Improvement of two points on the COPM reflects clinically significant improvement (“COPM Used in Research,” 2017. [Online]. Available: thecopm.ca). In addition to the COPM, the following standardized assessments were administered: the Action Research Arm Test (ARAT), which evaluated upper limb function by observing performance in a variety of tasks (N. Yozbatiran, L. Der-Yeghiaian, and S. C. Cramer, “A standardized approach toperforming the action research arm test,” Neurorehabil. Neural Repair, 2008.) and the Box and Blocks Test (BBT), which evaluated manual dexterity in having participants pick up and transport 2.5 cm wooden blocks over a 15.2 cm partition (V. Mathiowetz, G. Volland, N. Kashman, and K. Weber, “Adult norms for the Box andBlock Test of manual dexterity.,” Am J Occup Ther., vol. 39, no. 6, pp. 386-91, 1985.). The ARAT consists of 19 items that are grouped into categories of grasp (n=6 items), grip (n=4 items), pinch (n=6 items), and gross arm movements (n=3 items). Performance on each item is observed and rated on a 4-point scale ranging from 0 (no movement possible) to 3 (performs with normal movement). The total score on the ARAT ranges from 0 to 57, with the lowest score indicating no movements and the upper score indicating normal performance. For the BBT, the number of blocks acquired and transported in one minute is counted.

We also administered patient reported outcomes, namely the ABILHAND-Manual Ability Measure and Patient Reported Outcomes Measurement Information System (PROMIS) UE Short Form Version 2.0. The ABILHAND is a Rash-derived scale that has 23 tasks that are rated on a three-point difficulty scale (impossible, difficult, easy). Raw cores were converted into a linear score using the online program for conversion specific to chronic stroke (“Abilihand Score Conversion.” [Online]. Available: rssandbox.iescagilly.be). Higher ABILHAND scores indicate less difficulty completing the tasks (S. M. Gustafsson, K. S. Sunnerhagen, and S. Dahlin-Ivanoff, “Occupational Therapists' and Patients' Perceptions of ABILHAND, a New Assessment Tool for Measuring Manual Ability,” doi.org, vol. 11, no. 3, pp. 107-117, 2009.). PROMIS UE SF has 7 items that are rated on a 1-5 scale, where 1 is unable to do and 5 is without any difficulty (M. Hung, M. W. Voss, J. Bounsanga, A. B. Crum, and A. R. Tyser, “Examination of the PROMIS upper extremity item bank,” J. Hand Ther., vol. 30, no. 4, pp. 485-490, October 2017.).

We uploaded PROMIS SF scores to the HealthMeasures online scoring service (“PROMIS SF Conversion.” assessmentcenter.net) to convert them to the T-metric (mean=50, standard deviation=10).

### Results

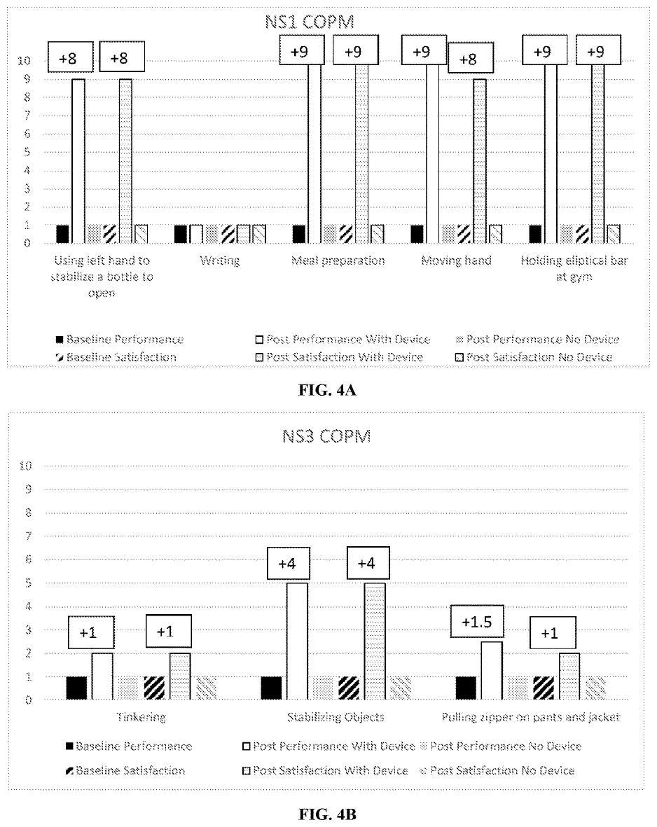

Both participants were successfully fitted with the customized NuroSleeve and participated in all 24 occupational therapy sessions over an 8-week period. NSAD01 and NSAD03 began using the NuroSleeve at home after the 18^{th} and 8^{th} therapy session, respectively. Performance of and satisfaction with each participant's COPM goal while using the NuroSleeve improved compared to baseline and to post-intervention without the device in all goals except one (writing for NS1; FIGS. 4A-B). Moreover, with the NuroSleeve, a clinically significant improvement (2:2 points) in both performance and satisfaction was demonstrated for each goal established by NS1, and for one of the three goals established by NS3. Baseline results with end of intervention scores are shown in Table 1. There was one adverse event and it was deemed unrelated to the intervention (breakthrough seizure due to known epilepsy).

TABLE 1
Baseline and Follow-Up Scores, With and Without NuroSleeve.

| | | | Follow-Up | | | |
|---|---|---|---|---|---|---|
| | | | Without | With | Change Score | |
| | | Baseline | NuroSleeve | NuroSleeve | Without | With |
| NS1 | ARAT | 1 | 3 | 9 | +2 | +8 |
| | BBT | 0 | 0 | 3 | 0 | +3 |
| | ABILHAND | 15 (0.368) | 20 (0.368) | 42 (0.702) | | |
| | Logit (SE) | | | | | |
| | PROMIS SF V2.0 | 28.8 (2.2) | 36.5 (2) | 35.3 (2) | | |
| | T-Metric (SD) | | | | | |
| NS3 | ARAT | 0 | 0 | 10 | 0 | +10 |
| | BBT | 0 | 0 | 6 | 0 | +6 |
| | ABILHAND | 7 (0.464) | 10 (0.417) | 23 (0.358) | | |
| | Logit (SE) | | | | | |
| | PROMIS SF V2.0 | 22.2 (3) | 31.5 (2.6) | 23 (2.8) | | |
| | T-Metric (SD) | | | | | |

Change scores are shown for the performance based measures (ARAT and BBT). ARAT = Action Reach Arm Test; BBT = Box and Block Test; SE = standard error (in logits); SD = standard deviation.

### Discussion

This report describes the results of an 8-week occupational therapy intervention utilizing a novel, lightweight, powered hand orthosis (NuroSleeve) in two people with chronic stroke. Both participants exhibited severe levels of UE impairment as quantified on the baseline measures. Both participants successfully learned how to utilize NuroSleeve for performance of activities and transitioned from using the NuroSleeve during supervised therapy to independent use at home. Both participants reported improvement in performance of, and satisfaction with, self-identified goals, as measured by the COPM, when the device was in use. The improvement on three of the goals chosen by NS1 and one of the goals identified by NS3, exceeded the two-point threshold for a clinically meaningful change. By the end of the 8-week intervention, both participants demonstrated improvements on the functional ARAT and BBT testing and the ABILMHAND measure, though only when the NuroSleeve was donned and in operation. This finding suggests the potential for the NuroSleeve in improving arm function and manual dexterity, even in severely compromised limbs. For NS1, PROMIS SF improvements were similar whether the NuroSleeve were in use or not, whereas for NS3, scores were in fact better without the NuroSleeve (even though ARAT and BBT scores improved and COPM goals were only achieved with it in use). This discrepancy may indicate that the different measures capture different aspects of the intervention, perhaps with the PROMI SF reflecting a mass practice benefit of the therapy and every-day at home arm use, rather than a specific benefit of the physical operation of the NuroSleeve. Another possibility is that the discrepancy is artifactual due to the details of the questions asked (e.g., the SF item about being able to wash one's back must be skipped because the NuroSleeve should not be used while bathing).

The version of the NuroSleeve described herein only addresses hand open-close function, and hence cannot be directly compared to the MyoPro (Myomo Inc, Cambridge, MA), which also has a motor to mobilize the elbow. The NuroSleeve is lighter (<½ lb versus >2 lb) and was anecdotally noted by both participants to be easier to don and doff than the forearm piece of the MyoPro. Another crucial distinction is the option of different control modes. The MyoPro only offers EMG sensing. While a myoelectric control strategy is logical for a person with limb amputation because the proximal residual limb is expected to be neurologically normal and intact, in our experience, individuals with upper motor neuron injury exhibit within- and across-day spontaneous variations in tone and EMG signals. Both the participants and the interventionist therapist found the mechanical switch and inertial measurement unit options much more practical, reliable, and functional than an using an EMG-based switch. In addition to the type of sensor, the therapist can also select the location of the sensor. Hence, if residual movement is not consistently available on the affected limb (e.g., proximal shoulder), then another site on the body can be used (e.g. contralateral shoulder shrug, ipsilateral foot tapping, etc.).

Using IMU and manual control, as opposed to EMG control, minimizes the amount of time required by the therapist to calibrate and set up the device operational parameters. Relying on an analog joystick and IMUs allowed the participants to use the NuroSleeve without the need for a connected computer, because the device settings did not need to be re-adjusted. Specifically, the NuroSleeve system required a quick (less than 2 minutes) set-up, and this was only needed only when the participant and the therapist wanted to switch to a different control strategy. Given the broad range of etiologies and severity of upper extremity impairment, these additional options in the type and location of input mechanisms may allow the approach to treatment to be tailored to the individual patient and their desires and needs.

The two participants and the occupational therapist interventionist provided useful feedback to inform improvements of the NuroSleeve system, these are summarized in Table 2.

TABLE 2
Modifications on the NuroSleeve design requested by
participants and the therapist- interventionist.

Minimize and where possible remove any dangling wires
Modify the hand piece such that the motor moves digits
2 to 5 rather than just digits 2 and 3
Switch from bespoke stainless steel hammered by a
human technician to a 3D-printing system to
accelerate fabrication and lower cost
Incorporate functional electrical stimulation as a
mobility option to complement the motorized
orthosis
Enable voice-activation to trigger hand open/close
Increase the hand aperture for grasping larger objects
Make the NuroSleeve a soft, flexible actual sleeve
rather than a rigid brace-orthosis

As an n-of-2 case report of an open-label intervention, there is not adequate statistical power to assert that the intervention had a significant benefit over mass practice without the device, or not having the device at all: even so, the fact that every tested outcome measure improved from baseline implies merit to that ongoing investigation. Furthermore, the ability to contrast functional performance, with and without the device, after the 8-week intervention, does help disambiguate the relative added effects of the device beyond the in-person occupational therapy intervention; it is not biologically plausible that these additive gains in functional movements would have occurred through chance alone, in particular in two individuals who had already exhausted multiple prior courses of outpatient occupational therapies only to remain at a plateau.

As of this writing, the manufacturer of the stainless-steel forearm brace offers a motorized version of their brace: that version is limited to a push-button trigger (requiring a stronger contralateral limb and hand). Hence the therapy regimen described in this report for the first participant (NS1) is in principle available to whomever acquires that off-the-shelf device, whereas incorporation of an inertial measurement unit as a trigger is not, to our knowledge, in any commercially available project. For any powered UE orthosis to be widely scalable to help more people with chronic UE functional impairments, financial barriers and technical support must be addressed and hand therapy specialists must become familiar with this as an option.

Another bottleneck to adoption is awareness: occupational therapists, physical therapists, physiatrists, and neurologists may simply not be aware that powered UE orthoses are available as an option or may see them only as a temporary rehabilitation training tool rather than a practical, everyday assistive device to enhance independent function at home.

In addition to demonstrating a promising trend of functional improvement, this case report also serves to demonstrate a promising interdisciplinary model where a team comprised of neurology, electrical engineering, software development, industrial design, and occupational therapy, can collaborate to make a durable, affordable, and genuinely functional assistive device.

### Conclusion

Two adults with UE mobility impairment due to stroke were able to learn to use a lightweight, customized, powered hand orthosis. This study suggests that the NuroSleeve system can be used to enable individuals with chronic UE impairments to independently perform valued daily activities in the home setting.

### Example 2

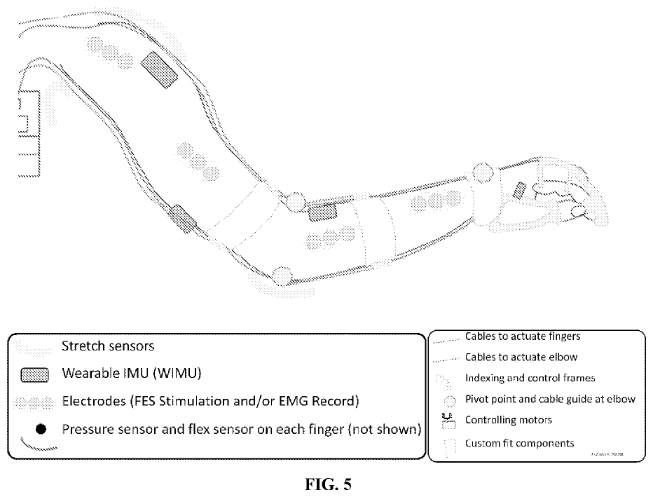

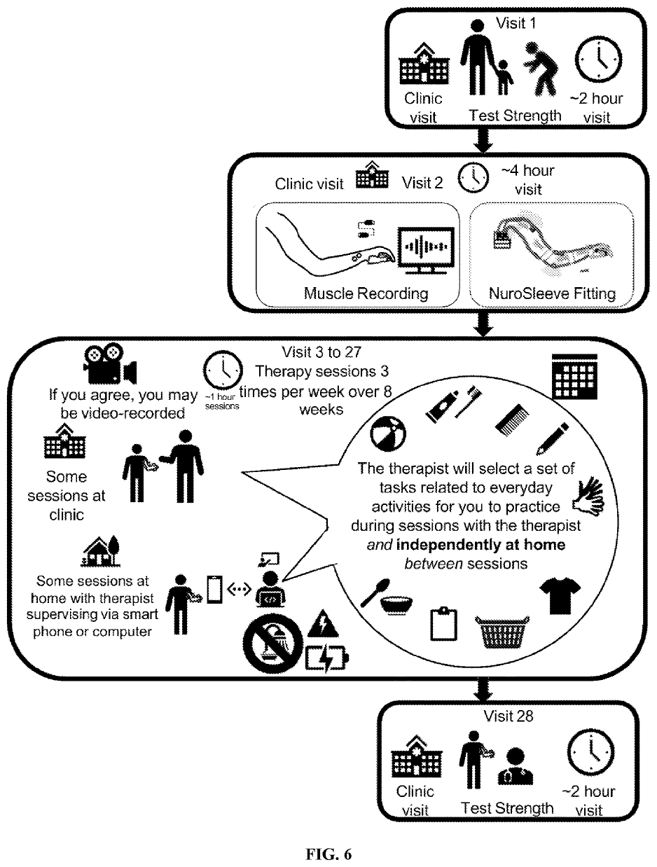

This Example shows an overview of how the NuroSleeve can function both as a therapeutic (restore mobility) and a diagnostic (log UE posture, joint angles, activity and use, contraction states etc). A schematic showing the elements of the device is illustrated in FIG. 5. To evaluate and demonstrate the feasibility of the device design the trial shown in the schematic of FIG. 6 was developed. The trial included 10 adults and 10 children with chronic UE impairment. The possible etiologies of the subjects included stroke, cerebral palsy, ALS, SMA, muscular dystrophy, arthrogryposis, spinal cord injury, brachial plexus injury, heritable neuropathies, peripheral nerve injury, and musculoskeletal injury.

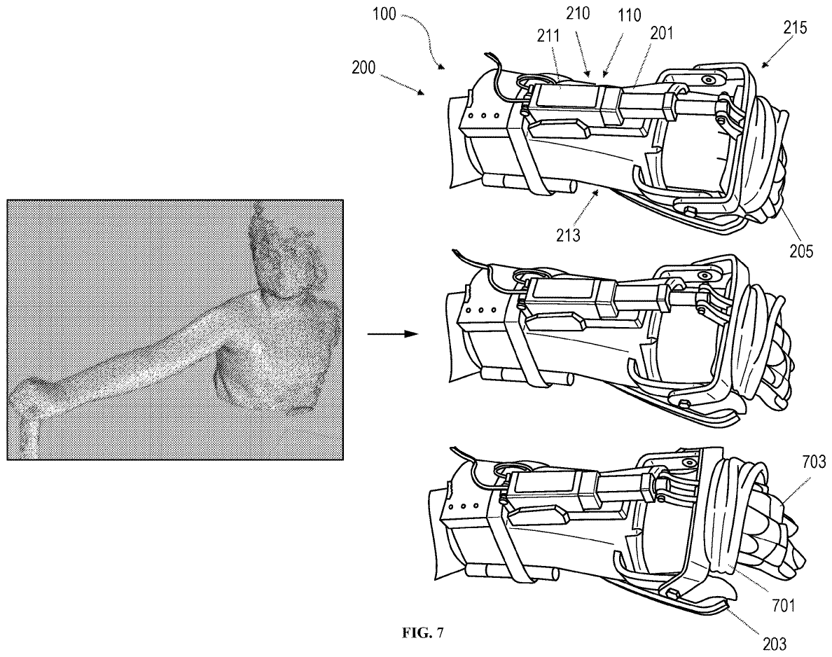

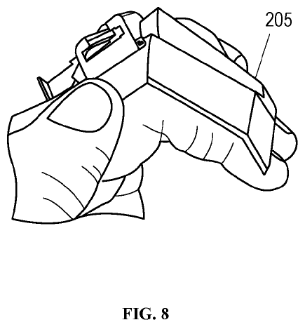

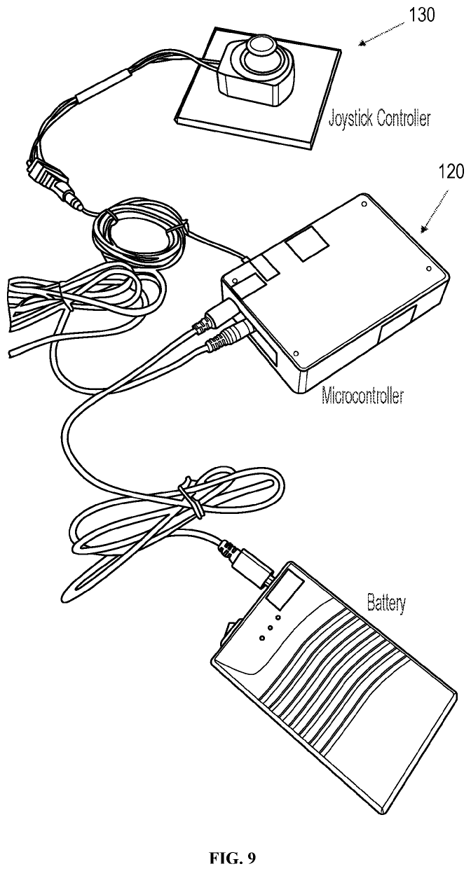

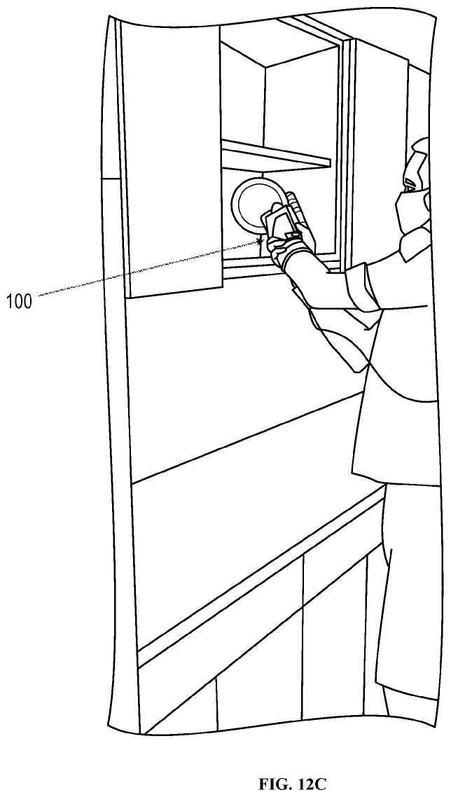

As illustrated in FIG. 7, a 3D scan of a person's forearm can be used to generate an orthotic brace, upon which a motor and microcontroller circuitry can be used to open and close the hand. Referring to FIG. 8, the device components may be 3D printed components tailored for the person's specific finger and hand anatomy. These components may be 3D printed from any suitable material, such as carbon fiber. Additionally, the device may optionally include certain off-the-shelf components such as metal screws. The motor, microcontroller, padding, straps, and/or IMU sensors are added to the 3D printed orthosis as needed, including the addition of the padding and straps easily be added by the therapist (within their scope of practice). Certain elements of the device (e.g., power source, FIG. 9) are electrically coupled to the device and held separately. Turning to FIG. 10, the carbon fiber 3D brace as discussed above is easy to don and doff independently by the subject. Functional electrical stimulation stickers and power may be further added to the device, facilitating additional movements that the brace-and-motor cannot achieve such as forearm supination. As illustrated in FIG. 11, a soft undersleeve can be used to line up the position of stimulator electrodes and sensors for some subjects, while cables may be routed along a soft sleeve for other subjects with flaccid (loose) weakness. Using the device described above, the subject was able to perform various desired tasks (FIGS. 12A-C).

The results from an evaluation of two subjects following an eight-week intervention involving use of the device at home to accomplish desired activities are shown in Tables 3-5 below. Both participants had hemiparesis due to chronic stroke. The study's outcomes are based on performance evaluation of validated measures of upper extremity function with and without wearing the NuroSleeve device. Outcomes were evaluated at baseline after enrollment, and again after completion of the eight-week intervention (three 45-minute sessions per week, for a total of 24 sessions).

TABLE 3
Abbreviated medical history of first two participants.

| | Participant Identification Number | |
|---|---|---|
| | NS1 | NS3 |
| Age bracket at enrollment | 40-55 | 50-55 |
| Years since stroke | 7 | 4 |
| Stroke history | Spontaneous right-sided basal | Right MCA stroke secondary |
| | ganglia hemorrhage with | to right ICA dissection and |
| | midline shift, requiring | M1 occlusion, required |
| | emergent decompressive | decompressive |
| | hemicraniectomy | hemicraniectomy |
| Primary control mode to | Joystick controlled by intact | Inertial measurement units |
| trigger device to achieve hand | right hand | donned on intact right |
| opening/closing | | shoulder |

Both participants developed chronic hemiparesis, primarily affecting the left upper extremity, due to right hemispheric stroke. Working with the trial team - incorporating feedback from both the occupational therapist interventionist and the engineering team - a variety of control modes were tested; the table shows the primary hand grasp strategy used to trigger the device.

TABLE 4
Performance on the Action Research Arm Test
(ARAT) and the Box and Blocks task.

| | Participants | | | | | |
|---|---|---|---|---|---|---|
| | NS1 | | | NS3 | | |
| Outcome Measures | Baseline | End | Change | Baseline | End | Change |
| ARAT (no device) | 1 | 3 | +2 | 0 | 0 | 0 |
| ARAT (with device) | N/A | 9 | +9 | N/A | 10 | +10 |
| Box & Blocks (no | 0 | 0 | 0 | 0 | 0 | 0 |
| device) | | | | | | |
| Box & Blocks (with | N/A | 3 | +3 | N/A | 6 | +6 |
| device) | | | | | | |

The ARAT measures a person's ability to grasp, grip, and pinch objects as well as measures gross movement. For the Box and Block test, a person has 1 minute to move as many individual 1-inch-per-side wooden cubes from one box into an adjacent box as possible.

TABLE 5
Performance scores on the Canadian Occupational Performance Measure (COPM).

| | Participants | | | | | | |
|---|---|---|---|---|---|---|---|
| | NS1 | | | | NS3 | | |
| | | End with | | | | End with | |
| | Baseline | device | Change | | Baseline | device | Change |
| Use left hand to | 1 | 9 | +8 | Tinkering | 1 | 2 | +1 |
| stabilize bottle to open | | | | | | | |
| Writing | 1 | 1 | 0 | Stabilizing | 1 | 5 | +4 |
| | | | | objects | | | |
| Meal preparation | 1 | 10 | +9 | Zipper on | 1 | 2.5 | +1.5 |
| | | | | pants & jeans | | | |

The COPM comprises a list of activities specifically prioritized by the participant. 1 = not able to do it at all, 10 = able to do it extremely well.

The trial preliminary results demonstrate that both participants exhibited improvements. The first participant (NS1) had previously used the MyoPro and found it too heavy and unwieldy to use; despite an earnest commitment to make it work for him this participant (a former Marine who completed two tours in Afghanistan), ultimately relegated the MyoPro to a closet. He relayed that he did not like the fact that he required the assistance of a family member to don and doff the MyoPro, and continually had to adjust settings with the laptop to make it have any type of benefit. In contrast, he is able to don and doff the NuroSleeve independently and wears it eight hours daily at home, to perform bimanual tasks such as opening bottles, food preparation, and opening envelopes.

Although these participants had hemiparesis due to stroke, it is believed that the device is suitable for use in subjects with other conditions, such as, but not limited to, people with UE impairment due to muscular dystrophy, spinal cord injury, and various other conditions.

### Example 3

This Example describes a powered orthosis, referred to herein as the NuroSleeve. The NuroSleeve is an innovative upper extremity 3D-printed orthosis that is custom-made for individuals living with disabilities, such as, but not limited to, adults and children diagnosed with long-term muscle weakness or partial paralysis. The NuroSleeve deploys a combination of motors and Functional Electrical Stimulation (FES), controlled via a series of wearable sensors, to alleviate mobility impairments and restore function and independence.

### Device Overview

The NuroSleeve includes an innovative orthosis design; an electronic Main Control Board (MCB) that contains all electronic components, including the microcontroller, the inputs (e.g., EMG, IMUs, joysticks, buttons and switches), and the control outputs (e.g., motors and FES); computer software to setup the device; and firmware that runs on the Main Control Board. Each orthosis is user-customizable and personalized to the user using a proprietary process that takes a 3D body scan of the user's upper extremity and turns into a custom 3D design for fabricating the orthosis, using additive manufacturing. Once the orthosis has been fabricated, the NuroSleeve leverages wearable sensors (2 or more), such as Inertial Measurement Units (IMUs), EMG, joysticks, buttons and switches to implement personalized and optimal control strategies for each user.

### Main Component Designs

### Orthosis Design

FIG. 13A shows a fully assembled CAD model of the 3D printed pieces of the NuroSleeve, without any of the other components installed. The other components, including hardware such a bolts, nuts, and pins; a linear actuator; a main control board; a battery; and functional electrical stimulation are added after the orthosis has been 3D printed (FIG. 13B).

The main elements the orthosis are a top arm piece where the motor mounts, a finger piece that the motor moves, a thumb piece that attaches to the arm piece via a hinge, and a bottom cuff piece that attaches to the same hinge. When powered, the motor moves the finger pieces in an open and close motion, allowing the user to grasp and release objects. The hardware components which are added to form the fully assembled device include, but are not limited to, a linear actuator having any suitable stroke length (e.g., a stroke length of two inches), a mounting bracket and pin for the motor, a headless clevis pin with retaining rings, three bolts and nuts, two bearings, a metal rod for the hinge, and straps with fasteners (e.g., hook and loop fasteners). The straps are used to keep the user's fingers and arm in place against the splint.

### Main Control Board Design

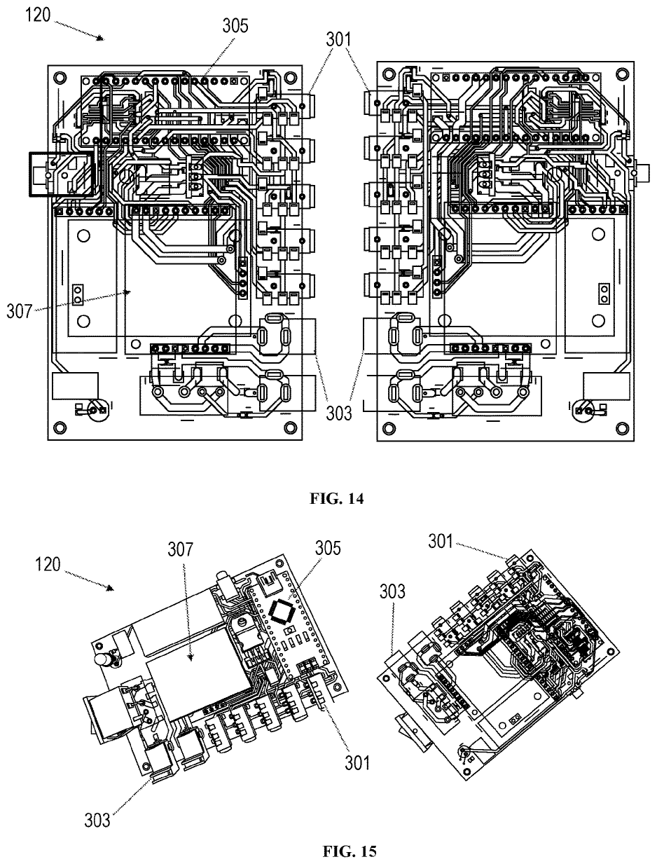

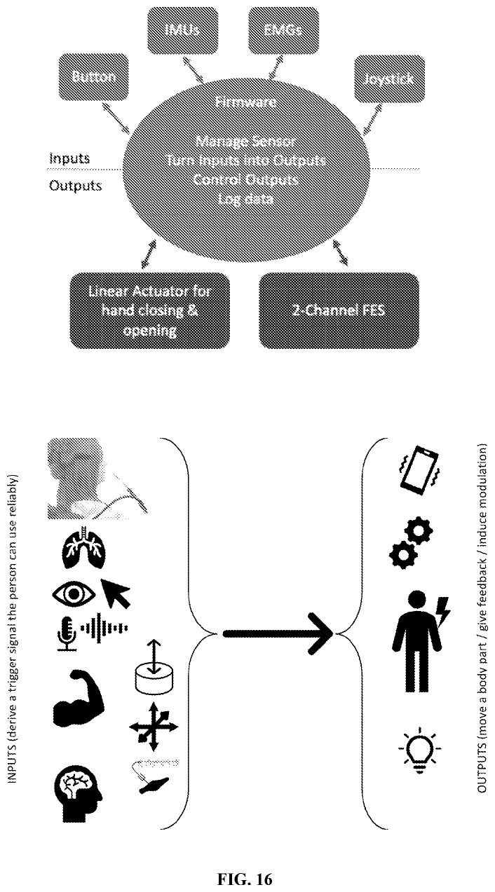

The Main Control Board (MCB), or Main Control Unit (MCU), includes a microcontroller, a wireless communication chip (e.g., Bluetooth chip), at least one motor driver, at least one input connector (e.g., connectors for two IMUs, two EMGs, a joystick, a switch, four buttons, and a voice recognition module) and at least one output connector (e.g., linear actuator, FES), and a memory chip. Examples of a printed circuit board (PCB) and MCB are shown in FIGS. 14 and 15, respectively.

### Firmware

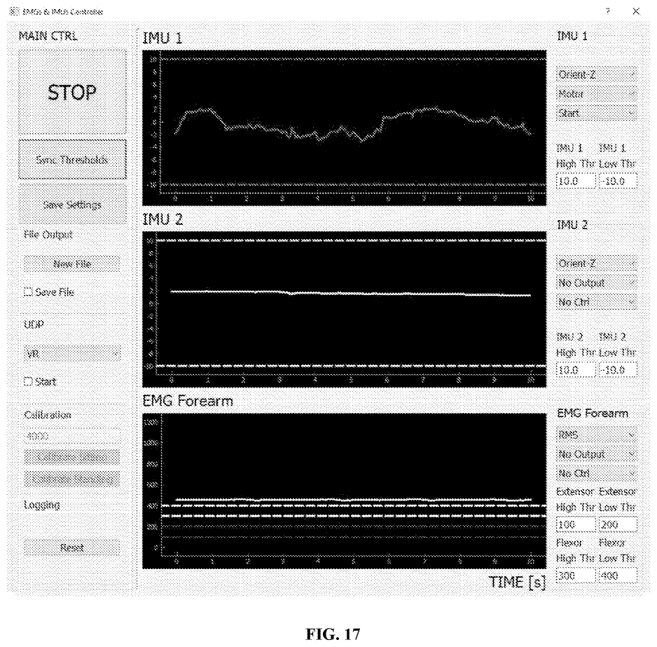

The device embedded firmware manages all the input sensors and utilizes user selected control strategies to turn the inputs into control outputs for the linear actuator that assists with hand closing and opening and a third-party external 2-channel FES device that can be used to stimulate different UE muscle groups. Furthermore, the firmware can store on-board user usage and establish a bidirectional communication with the accompanying software. FIG. 16 illustrates an example of the firmware input/output mapping.

With respect to the inputs, the EMG input is interchangeable with any surface, physiological signal. For example, if one places the same exact stickers-sensor used for EMG on the chest, one gets an Electrocardiogram, while if placed lateral to the eye, one would pick up an Electro-oculogram. The microcontroller can take the EMG input signal from any suitable positioning on the subject and use it to trigger an output. The signal could also simply be the skin itself (skin conductance). In addition to those shown in FIG. 16, various other inputs can be used. For example, although not shown, voice recognition input has been implemented and demonstrated in the device.

Turning to the outputs, the motor may be a linear actuator or another type of motor. The motor(s) may open/close the hand and/or provide any other suitable motion (e.g., flex/extend the elbow) depending upon how it is mounted on the orthosis. Additionally, the output is not limited to inducing functional movement and may include, but is not limited to, a vibrotactile element (a motor with an eccentric mass or a tiny diaphragm; much like smart phones can vibrate and buzz in silent mode), turn off and on an indicator light, play a sound file etc.

### Software

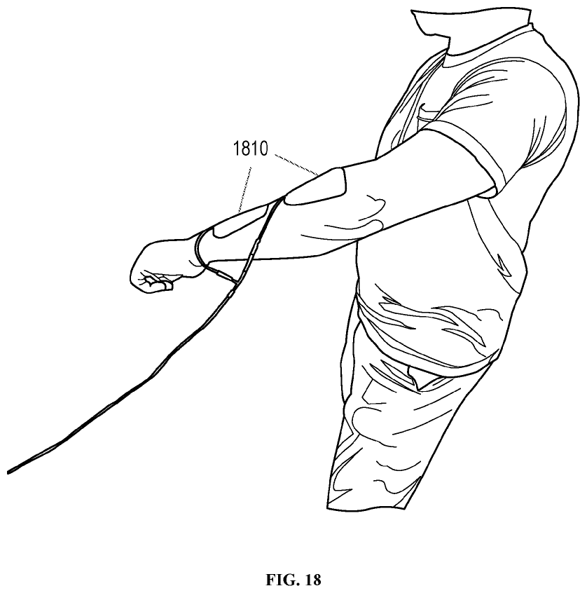

The NuroSleeve software has been developed to run on a Windows PC and can connect to the NuroSleeve firmware via Bluetooth. Although designed for a Windows PC, the software can be modified to run on any suitable operating system. The software serves three main purposes 1) Device setup and calibration; 2) Sensor data visualization and acquisition; and 3) External control of NuroSleeve. An image of the software user interface is shown in FIG. 17.

### Example 4

This Example describes a shoulder assist device with bands as mobility assistive elements.

### Intended Use

The device is an upper extremity 3D-printed orthosis that is custom-made for each user, depending on their individual physical characteristics and capabilities. The device aids users in using their upper extremity

### Indications for Use

The device is indicated for use by adults and children diagnosed with long-term muscle weakness or partial paralysis. Users must meet physical size specifications and demonstrate capacity to use the device, including sufficient cognitive abilities, per user agreement and clinician evaluation.

### Description

The device is composed of several linked pieces, the number of which depends on the size of the user's upper extremity and the wheelchair or mounting solution. For example, the device may have an arm hold, for the user to rest their arm in, a forearm/elbow joint that allows for internal rotation, rods that pivot allowing for shoulder flexion and extension, a shoulder piece that allows for internal rotation at the shoulder level, and back support pieces that vary depending on the user's chair. FIGS. 19A-C show 3D printed components of a device for a left arm, without the hardware or the rubber bands used as support. The hardware not shown in FIGS. 19A-C includes ball bearings, bolts, nuts, hook and loop fasteners, and fabric used to cushion the arm pad and keep the user's wrist in the device. Additionally, 3D printed components used to hold the rubber bands are not included. A distal portion of the device is not illustrated in FIGS. 19A-C, as is it variable depending on the wheelchair.

The individual building blocks of the device can be interchanged depending on the user's arm length and needs. In the set up illustrated, the blocks allow for rotation on the wrist piece down, internal rotation of the elbow, and internal rotation of the shoulder in one piece, however with different pieces joints could be added or removed. Turning to FIG. 19D, once assembled, rubber bands are attached to the building blocks at the ends of the rods, as well as at the holes near the forearm pieces, to support both the proximal and distal arm. Finally, a motor piece can optionally be added to drive the internal rotation of the elbow joint for users that need additional support.

The rubber bands that control/modulate shoulder flexion and extension assistance may be engaged and disengaged as desired. In other words, at the push of a button, the amount of support provided by the rubber bands in helping the user to lift or to lower their arm can be modified instantly. In contrast to existing devices, where user's continue to struggle in performing various tasks as they have to constantly push to overcome the device effects, the device described in this Example allows the user to effortlessly keep their arm down on a table or lift it up based on the desired physical activity. In addition, the device described in the Example provides increased mechanical stability over existing devices, adjustable mounting capabilities (e.g., the device can be mounted on several wheelchair models, using adjustable and custom (3D-printed) mounts), a larger range of motion compared to existing devices, and a lightweight and portable design. Furthermore, depending on user needs the device can be set up to operate in two modes: 1) rubber bands to support both shoulder flexion/extension and wrist flexion/extension; 2) rubber bands only to support shoulder flexion/extension while an electric motor helps with wrist flexion/extension. This is highly beneficial for some users who cannot achieve enough wrist degrees of flexion to perform activities of daily living and/or also provides better control for hand movements.

The disclosures of each and every patent, patent application, and publication cited herein are hereby incorporated herein by reference in their entirety.

### INCORPORATION BY REFERENCE

The entire contents of all patents, published patent applications, and other references cited herein are hereby expressly incorporated herein in their entireties by reference.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An upper extremity mobility assistive device, comprising:
a main control board;
at least one input article coupled to an input of the main control board; and
at least one mobility assist element coupled to an output of the main control board.

2. The device according to claim 1, further comprising an orthotic brace, the orthotic brace including:
an arm portion substantially mirroring the shape of a user's forearm;
a thumb portion coupled to the arm portion; and
a finger portion movably coupled to the arm portion.

3. The device according to claim 2, wherein the arm portion comprises a top piece and a bottom piece.

4. The device according to claim 3, further comprising a hinge coupling the bottom piece to the top piece in a clamshell fashion.

5. The device according to claim 4, wherein the thumb portion is coupled to the top piece through the hinge.

6. The device according to claim 4, further comprising a second hinge coupling the thumb portion to the arm portion.

7. The device according to claim 2, wherein the thumb portion is adjustable relative to the arm portion.

8. The device according to claim 3, further comprising a hinge movably coupling the finger portion to the top piece.

9. The device according to claim 8, wherein the hinge coupling the finger portion to the top piece is non-orthogonal relative to the long axis of the user's forearm.

10. The device according to claim 8, wherein the hinge coupling the finger portion to the top piece is tilted in two planes relative to the long axis of the user's forearm.

11. The device according to claim 2, wherein the finger portion supports all four of a user's fingers together.

12. The device according to am claim 2, wherein the finger portion leaves a user's fingertips exposed.

13. The device according to claim 2, wherein the arm portion includes at least one opening leaving a bony prominence on a user uncovered or unimpinged upon.

14. The device according to claim 13, wherein the bony prominence is selected from the group consisting of the elbow, the scapula, the clavicle, the medial side of the wrist, the lateral side of the wrist, the knuckles, and combinations thereof.

15. The device according to claim 1, wherein the main control board comprises:
a microcontroller;
a Bluetooth chip;
a motor driver;
at least one input connector;
a memory chip; and
embedded firmware.

16. The device according to claim 1, wherein the at least one input article is selected from the group consisting of an inertial measurement unit (IMU), an electromyography (EMG), a joystick, a switch, a button, a voice recognition module, and combinations thereof.

17. The device according to claim 1, wherein the at least one mobility assist element comprises a motor coupled to the arm portion and the finger portion.

18. The device according to claim 17, further comprising a linear actuator coupling the motor to the finger portion.

19. The device according to claim 1, wherein the at least one mobility assist element comprises a stimulation article.

20. The device according to claim 19, wherein the stimulation article is selected from the group comprising mechanical vibration, transcutaneous electrical neural stimulation (TENS), functional electrical stimulation (FES), other two-channel electrical stimulation, and combinations thereof.

21. The device according to claim 19, wherein the stimulation article comprises a functional electrical stimulation (FES) article.

22. The device according to claim 21, wherein the FES article is arranged and disposed to induce targeted contraction of one or more muscle's in a subject's extremity, the targeted contraction effecting movement of the subject's extremity.

23. The device according to claim 19, wherein:
the stimulation article includes at least two stimulation articles;
at least one of the stimulation articles is arranged and disposed for positioning on the subject's extremity; and
at least one of the stimulation articles is arranged and disposed for positioning over the subject's spine.

24. The device according to claim 19, further comprising software configured to direct activation of the stimulation article.

25. The device according to claim 24, wherein the software is configured to set parameters of the embedded firmware.

26. The device according to claim 25, wherein the software sets the parameters of the embedded firmware to simultaneously activate the at least one stimulation articles arranged and disposed for positioning on the subject's extremity and the at least one stimulation articles arranged and disposed for positioning over the subject's spine.

27. The device according to claim 23, wherein at least one of the stimulation articles is wireless.

28. A method of restoring movement of a user's extremity, the method comprising:
fitting the orthosis device according to claim 1 to the user's extremity;
securing the orthosis device to the user's extremity;
receiving at least one input signal at the main control board of the device; and
generating at least one output to move the user's extremity.

29. The method of claim 28, wherein the at least one output is selected from the group consisting of a powered output through the motor, a stimulation output through the stimulation article, and a combination thereof.

30. A method of stimulating movement of a user's extremity, the method comprising:
positioning at least one of the stimulation articles according to claim 23 on the user's extremity;
positioning at least one of the stimulation articles according to claim 23 over the user's spine;
receiving at least one input signal at the main control board of the device; and
generating at least one output to stimulate the user's extremity;
wherein the at least one output activates both the at least one stimulation article on the user's extremity and the at least one stimulation article on the user's spine; and
wherein the activation of both the at least one stimulation article on the user's extremity and the at least one stimulation article on the user's spine synergistically stimulates the user's extremity.
